(19) United States
(12) Patent Application Publication
(10) Pub. No.: US 2010/0010450 A1
(43) Pub. Date: Jan. 14, 2010

(54) RETRACTABLE SYRINGE WITH IMPROVED STEM RING AND NEEDLE INTERCHANGEABILITY

(76) Inventors: Vincent Runfola, Apopka, FL (US); John J. Bojan, Colorado Springs, CO (US)

Correspondence Address: EDELL, SHAPIRO & FINNAN, LLC, 1901 RESEARCH BOULEVARD, SUITE 400, ROCKVILLE, MD, 20850, US

(21) Appl. No.: 12/482,697

(22) Filed: Jun. 11, 2009

Related U.S. Application Data
(60) Provisional application No. 61/061,224, filed on Jun. 13, 2008.

(51) Int. Cl. A61M 5/31; A61M 5/315; A61M 5/34; A61M 5/32; B23P 11/00

(52) U.S. Cl. 604/190; 604/218; 604/240; 604/192; 29/428

(57) ABSTRACT

A syringe includes a needle assembly at least partially secured within a barrel of the syringe, the needle assembly including a needle stem, a resilient member biasing the needle stem toward a proximal end opening of the barrel, a needle holder connectable to the needle stem during assembly of the syringe and that is further securable to a needle, and a cap that is dimensioned to surround the needle secured to the needle holder and a portion of the needle holder during assembly of the needle holder with the needle stem. In another embodiment, a syringe includes a needle assembly including a section secured within the barrel, the needle assembly including a needle holder, a resilient member biasing the needle holder toward a proximal end opening of the barrel, and a retaining member releasably secured to the needle holder, where the retaining member includes a first material that connects the retaining member to the needle holder and a second material that engages an interior wall section of the barrel, the second material having a hardness or durometer value that is less than that of the first material.

### CROSS REFERENCE TO RELATED APPLICATIONS

This application claims priority from U.S. Provisional Patent Application Ser. No. 61/061,224, entitled “Retractable Syringe With Improved Stem Ring and Needle Interchangability”, and filed Jun. 13, 2008. The disclosure of this patent application is incorporated herein by reference in its entirety.

### BACKGROUND OF THE INVENTION

1. Field

The present invention pertains to safety medical syringes and, in particular, to syringes that are limited to a single use.

2. Description of the Related Art

Retractable and/or other types of single use syringes have become increasingly important and desirable for use in hospitals and medical facilities for a number of reasons. In retractable syringes, a hypodermic needle of the syringe is typically withdrawn into the syringe after aspiration and injection of a fluid, thus preventing multiple uses of the syringe and the potential transmission of human immunodeficiency virus (HIV) as well as other diseases from patient-to-patient. The retraction of the needle within the syringe after use also shields the needle and prevents inadvertent needle sticks from occurring to patients and health care providers.

A variety of different retractable or other types of single use syringe devices have been designed to effectively withdraw the needle within the syringe after use. For example, one example of a retractable syringe that is effective in ensuring a single use is described in co-pending U.S. patent application Ser. No. 11/249,741, the disclosure of which is incorporated herein by reference in its entirety.

The challenge exists to continuously improve upon the retractable syringe technology to design a single use syringe that is simplistic in design and assembly without sacrificing safety of the syringe, where the syringe can be produced on a large production scale while minimizing manufacturing costs.

### SUMMARY OF THE INVENTION

In one example embodiment, a syringe comprises a hollow barrel including a proximal end with an opening and a distal end configured to receive a syringe needle, wherein a portion between the proximal and distal ends of the barrel defines a fluid cavity to receive and expel fluid from the barrel distal end, a plunger extending into the proximal end opening of the barrel and axially movable within the barrel toward and away from the barrel distal end, wherein the plunger includes a proximal end and a distal end that extends within the barrel and is movable toward and away from the barrel distal end to facilitate drawing fluid into and forcing fluid from the fluid cavity within the barrel, and a needle assembly that is partially secured within the barrel, the needle assembly including a needle holder, a resilient member biasing the needle holder toward the proximal end opening of the barrel, and a retaining member releasably secured to the needle holder, wherein the retaining member includes a first material that connects the retaining member to the needle holder and a second material that engages an interior wall section of the barrel, the second material having a Shore A or Shore D durometer hardness value that is less than a Shore A or Shore D durometer value of the first material.

In another example embodiment, a syringe comprises a hollow barrel including a proximal end with an opening and a distal end configured to receive a syringe needle, wherein a portion between the proximal and distal ends of the barrel defines a fluid cavity to receive and expel fluid from the barrel distal end, a plunger extending into the proximal end opening of the barrel and axially movable within the barrel toward and away from the barrel distal end, wherein the plunger includes a proximal end and a distal end that extends within the barrel and is movable toward and away from the barrel distal end to facilitate drawing fluid into and forcing fluid from the fluid cavity within the barrel, and a needle assembly at least partially secured within the barrel, the needle assembly including a needle stem, a resilient member biasing the needle stem toward the proximal end opening of the barrel, a needle holder connectable to the needle stem during assembly of the syringe and that is further securable to a needle, and a cap that is dimensioned to surround the needle secured to the needle holder and a portion of the needle holder during assembly of the needle holder with the needle stem.

In a further example embodiment, a needle assembly is provided for use with a syringe comprising a hollow barrel including a proximal end with an opening and a distal end configured to receive a syringe needle, a portion between the proximal and distal ends of the barrel defining a fluid cavity to receive and expel fluid from the barrel distal end, a plunger extending into the proximal end opening of the barrel and axially movable within the barrel toward and away from the barrel distal end, the plunger including a proximal end and a distal end that extends within the barrel and is movable toward and away from the barrel distal end to facilitate drawing fluid into and forcing fluid from the fluid cavity within the barrel, and a needle stem and a resilient member biasing the needle stem toward the proximal end opening of the barrel. The needle assembly comprises a needle holder that is connectable to the needle stem during assembly of the syringe and that is further securable to a needle, and a cap that is dimensioned to surround the needle secured to the needle holder and a portion of the needle holder during assembly of the needle holder with the needle stem such that the needle is shielded by a portion of the cap when the needle holder with needle is secured to the needle stem of the syringe.

In still another embodiment, a method of securing a needle to a syringe is provided, the syringe comprising a hollow barrel including a proximal end with an opening and a distal end configured to receive a syringe needle, a portion between the proximal and distal ends of the barrel defining a fluid cavity to receive and expel fluid from the barrel distal end, a plunger extending into the proximal end opening of the barrel and axially movable within the barrel toward and away from the barrel distal end, the plunger including a proximal end and a distal end that extends within the barrel and is movable toward and away from the barrel distal end to facilitate drawing fluid into and forcing fluid from the fluid cavity within the barrel, and a needle stem and a resilient member biasing the needle stem toward the proximal end opening of the barrel. The method comprises installing a needle holder including a needle secured to the needle holder within a cap such that the cap shields the needle, and securing the needle holder to the needle stem of the syringe while the cap is shielding the needle.

The invention facilitates easier assembly of the needle stem including the two-piece or two-material retaining member within the barrel of the syringe and maintaining a fluid tight seal at the engagement between the retaining member and the interior of the barrel. In addition, the needle assembly facilitates easy needle interchangeability with the syringe while still allowing for needle retraction after a single use of the syringe to inject fluids within a patient.

The above and still further examples, features and advantages of the present invention will become apparent upon consideration of the following detailed description of a specific embodiment thereof, particularly when taken in conjunction with the accompanying drawings wherein like reference numerals in the various figures are utilized to designate like components.

### BRIEF DESCRIPTION OF THE DRAWINGS

FIG. 1 is a side view in cross-section of a syringe in accordance with an embodiment of the present invention, where the needle extends from the syringe barrel and the syringe is ready for use.

FIG. 2 is a partial view in perspective and partial section of the syringe of FIG. 1 including a proximal end portion of the syringe barrel.

FIG. 3 is an exploded view in perspective of a portion of the syringe of FIG. 1 including the needle assembly and a distal end portion of the syringe barrel with a section removed to reveal the barrel interior.

FIG. 3a is a side view of an alternative embodiment of a needle stem and a side view in cross-section of an alternative embodiment of a barrel for the retractable syringe of FIG. 1.

FIGS. 4-7 are partial side views in cross-section of the syringe of FIG. 1 detailing interaction of the distal end of the plunger and the proximal end of the needle assembly at varying stages of depression of the plunger to facilitate retraction of the needle assembly into the syringe in accordance with the present invention.

FIG. 8 is a side view in cross-section of the syringe of FIG. 1 with the needle fully retracted into the syringe after use.

FIG. 9 is a partial side view in cross-section of a retractable syringe including a needle hub configuration that facilitates needle interchangeability in accordance with the present invention.

FIG. 10 is a partial side view in cross-section of a retractable syringe including a needle hub configuration that facilitates needle interchangeability in accordance with another embodiment of the present invention.

FIG. 11 is a side view in cross-section of the needle hub connected with the needle stem for the retractable syringe of FIG. 10.

FIG. 12 is a side view in cross-section of a needle cap for the retractable syringe of FIG. 10.

FIG. 13 is a side view in cross-section of a needle hub for the retractable syringe of FIG. 10.

FIG. 14 is a side view in cross-section of the needle stem for the retractable syringe of FIG. 10.

FIG. 15 is a partial side view in cross-section of the needle hub connected with the needle stem inside the barrel of the retractable syringe of FIG. 10.

FIG. 16 is a partial side view in cross-section of a retractable syringe including a needle hub configuration that facilitates needle interchangeability in accordance with a further embodiment of the present invention.

FIG. 17 is a side view in cross-section of a base forming a first part of a needle cap for the retractable syringe of FIG. 16.

FIG. 18 is a partial side view in cross-section of a retractable syringe including a needle hub configuration and a fill spike that facilitates needle interchangeability in accordance with still another embodiment of the present invention.

FIG. 19 is a magnified partial side view in cross-section of the retractable syringe of FIG. 18.

FIG. 19A is a partial side view in cross-section of a modified version of the retractable syringe of FIG. 18, in which the base portion of the needle cap threadingly engages with the barrel of the syringe.

FIG. 20 is a partial side view in cross-section of a retractable syringe including a needle hub configuration that facilitates needle interchangeability in accordance with another embodiment of the present invention.

FIGS. 21A and 21B are side views in cross-section of a needle stem for a retractable syringe in accordance with still another embodiment of the present invention, in which the needle stem includes a fill spike that is removable to render the needle stem for use with an interchangeable needle.

FIG. 22 is a partial side view in cross-section of a retractable syringe including a needle hub configuration that facilitates needle interchangeability in accordance with another embodiment of the present invention.

FIG. 23 is a partial side view in cross-section of a retractable syringe including a needle hub configuration that facilitates needle interchangeability in accordance with a further embodiment of the present invention.

FIG. 24 is a partial side view in cross-section of a retractable syringe including a needle hub configuration that facilitates needle interchangeability in accordance with another embodiment of the present invention.

FIG. 25 and are side views in cross-section of different retractable syringes in accordance with the invention including a needle hub configuration that facilitates needle interchangeability and including a ring member disposed between a portion of the needle hub and the needle cap.

FIG. 27 is a side view of a needle hub and a side view in cross-section of a needle cap for a retractable syringe that facilitates needle interchangeability in accordance with an embodiment of the present invention.

### DETAILED DESCRIPTION

In accordance with the present invention, a single use retractable syringe is provided that facilitates easy retraction of portions of a needle assembly within the barrel of the syringe and also maintains a fluid tight seal between components within the syringe during use. An example embodiment of a single use retractable syringe of the invention is described below.

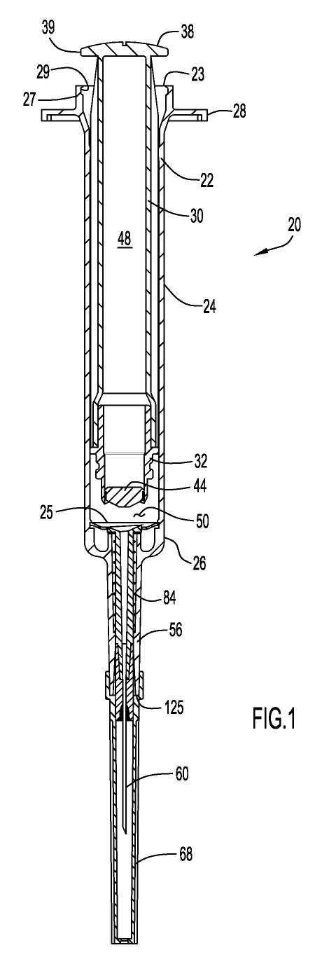

Referring to FIG. 1, a medical syringe 120 includes a hollow cylindrical barrel 122 with an opening 123 at its proximal end and suitably dimensioned to receive a hollow plunger 130. The barrel further includes an opening at its distal end to permit exposure of a needle 160 from the syringe. The barrel 122 includes a main body portion 124 that receives and retains a portion of the plunger 130 and a distal end extension 126 of reduced internal diameter in relation to the main body portion 124 that receives a needle assembly 156 as described below. At least one bump or ridge 212 is disposed along an interior surface wall portion of the main body portion 124 within and near the proximal end of the barrel to resist or prevent complete removal of the plunger from the barrel during operation of the syringe. For example, the ridge could be continuous (e.g., forming a protruding annular ring) within the barrel. Alternatively, a series (e.g., two or more) of ridges can be provided at radially spaced locations along the interior barrel surface.

The plunger 130 includes a resilient seal 132 encircling the plunger near its distal end. A fluid cavity 150 is defined within the barrel 122 between the resilient seal 132 and other distal end portions of the plunger and a distal end 125 of the barrel main body portion 124, where the fluid cavity varies in volume based upon axial displacements of the plunger with respect to the barrel. A sheath 168 is removably secured to the distal end of the barrel 122 to enclose needle 160 secured within the barrel prior to use of the syringe. The barrel, plunger, resilient seal, sheath and all other components of the syringe may be constructed of any suitable medical grade materials (e.g., plastics and/or stainless steels) that facilitate operability of the syringe as described below. Further, the syringe can be designed with a suitable fluid cavity to meet any fluid volume capacity for a particular application (e.g., 1 cubic centimeter or cc, 3 cc, 5 cc, 10 cc, etc.).

The proximal end of plunger 130 includes a convex surface or domed thumb pad 138 and a radially extending flange 139 that facilitates engagement with the fingers and/or thumb of the user during operation of the syringe. Similarly, the main body portion 124 of the barrel includes a radially extending flange 128 disposed near its proximal end for facilitating engagement with the fingers and/or thumb of the user during operation. An extended barrel portion 127 extends between flange 128 and the proximal end of the barrel and is slightly greater in internal diameter in comparison to the remainder of main body portion 124. The extended barrel portion 127 is also of a sufficient longitudinal dimension, and is slightly smaller in internal diameter than the transverse dimension of the plunger defined at the flange 139. When the plunger 130 is fully depressed within the barrel, an outer peripheral portion of the plunger flange 139 can be designed to flex or bend slightly so as to permit the flange to enter the extended barrel portion. Alternatively, the barrel can be designed to flex slightly so as to permit entry of the plunger flange within the barrel.

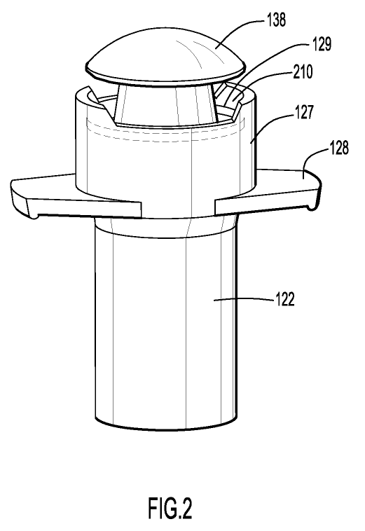

Optionally, two diametrically opposed sections can be removed from extended barrel portion 127, as shown in FIG. 2, so as to form cut-out areas or portions 210 along the extended barrel periphery. The cut-out portions 210 facilitate exposure of sufficient portions of the plunger thumb pad 138 and flange 139 to assist the user of the syringe in gripping the plunger when the plunger is pulled from the barrel to initiate aspiration of fluid within the fluid cavity of the barrel as described below. While two cut-out sections are depicted, it is noted that any suitable number of cut-out sections can be provided (e.g., one or more) at any one or more selected locations along the extended barrel portion. In addition, the extended barrel portion wall thickness and/or plastic or other materials from which this portion is formed can be selected to facilitate a slight elastic and reversible deformation of the extended barrel portion when grasped by a user so as to further assist in axially displacing a portion of the plunger from the barrel during aspiration of fluid into the barrel. However, it is noted that no cut-out sections are required to facilitate forcing of the plunger thumb pad within the barrel in the manner described below.

An annular groove 129 is disposed along interior wall sections (e.g., wall sections that are separated by the cut-out sections) of the extended barrel portion near the proximal end of the barrel. Upon complete depression of the plunger within the barrel and retraction of the syringe within the plunger, the plunger flange 139 engages in a snap-tight locking relationship with annular groove 129 to prevent removal of the plunger from the barrel.

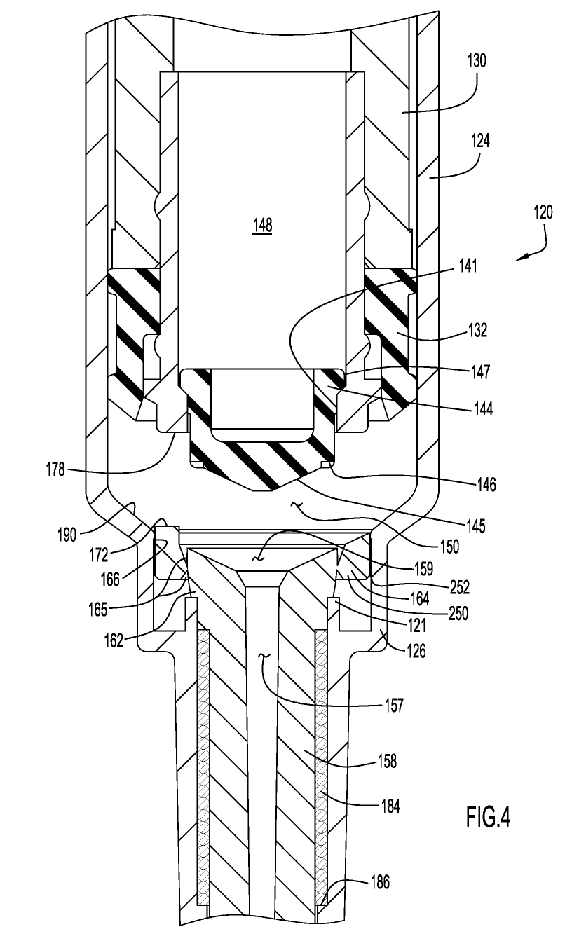

Referring to FIG. 4, plunger 130 includes a membrane or plug 144 disposed at a distal end of the plunger and that is frictionally held between interior wall portions of the plunger. The plug 144 seals the hollow interior or retraction cavity 148 of plunger 130, with the frictional engagement between the plug and the plunger being suitable to maintain engagement of the plug with the plunger until retraction of the needle assembly occurs as described below. Alternatively, it is noted that the plunger membrane or plug can be secured at the distal end of the plunger in a snap tight fitting relationship or in any other suitable manner.

As can be seen in FIG. 4, the distal end of plunger 130 includes an opening that communicates with retraction cavity 148 and into which plug 144 is secured. The interior annular wall at the distal end of the plunger includes an inwardly extending radial ledge 141. The diameter or transverse cross-section of the plunger opening, as defined at the ledge 141, is smaller than the diameter or transverse cross-section of the retraction cavity 148 that is defined within the plunger and lies beyond the ledge 141. Similarly, plug 144 includes an extending portion 147 that extends transversely from a proximal end of the plug. The extending portion 147 of the plug 144 is slightly larger in transverse cross-sectional dimension than the transverse cross-sectional dimension of the plunger opening defined at the ledge 141, so as to facilitate frictional contact between the ledge 141 and the extending portion 147 of the plug 144. The transverse cross-sectional dimensions of the extending portion 147 as well as the rest of the plug 144 are smaller than the transverse cross-sectional dimension of the retraction cavity 148 between the plunger ledge 141 and the proximal end of the plunger. Further, the transverse cross-sectional dimension of the remaining plug portion that extends between the extending portion 147 and the distal end of the plug 144 is slightly smaller than the transverse cross-sectional dimension of the plunger opening. Thus, upon axial displacement of the extending portion 147 of the plug 144 from the ledge 141 of the plunger 130 during retraction of the needle assembly, the frictional engagement between the plunger and the plug is released and the plug is free to move into the retraction cavity.

The plunger plug 144 includes a convex and frusto-conical surface 145 that extends toward the distal end of the barrel and engages with a generally complimentary, concave and frusto-conical cavity portion 159 of needle assembly 156 when the plunger is fully depressed into the barrel as described below. As noted in the previous embodiments described above, the end wall may alternatively be formed with any suitable outwardly or inwardly extending surface (e.g., conical, convex, V-shaped, multifaceted, etc.) or even a flat or planar surface as desired for a particular application. However, complimentary engaging surface features of the plunger plug and the needle assembly are preferred in order to minimize or eliminate open or “dead” space within the fluid cavity during removal of fluid from the fluid cavity.

At least one notch 146 is formed on the plug at the base of the frusto-conical surface. The notch 146 can extend around the periphery of the plug at the base of the frusto-conical surface or, alternatively, consist of a single notch or one or more spaced notch sections. The notch basically serves to provide a fluid flow path between the fluid cavity within the barrel and the fluid channel in the needle assembly when the plunger is substantially or completely engages with the needle assembly. In addition, the notched plunger plug minimizes or eliminates the potential for an increase or build-up of hydraulic pressure within the fluid cavity during movement of the plunger toward the needle assembly.

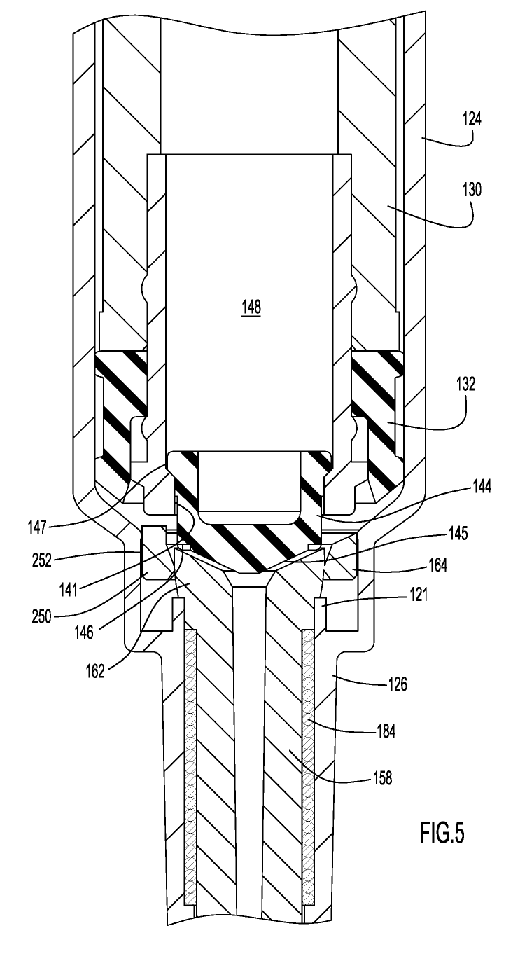

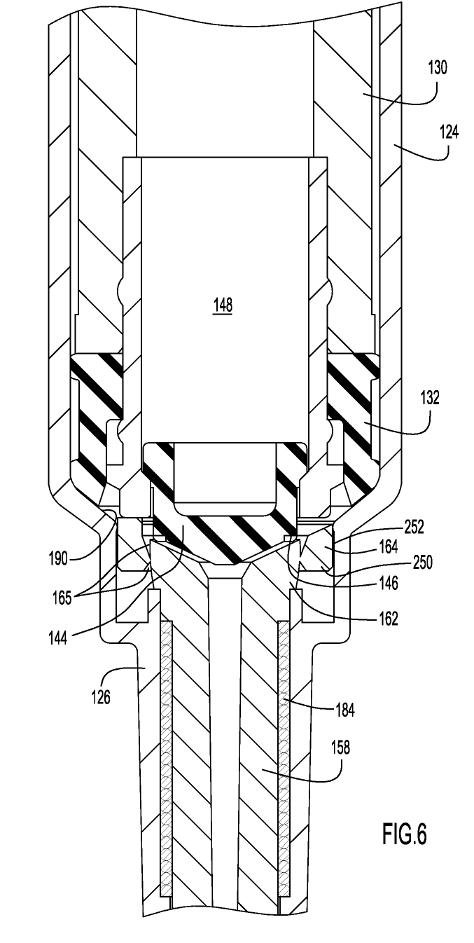

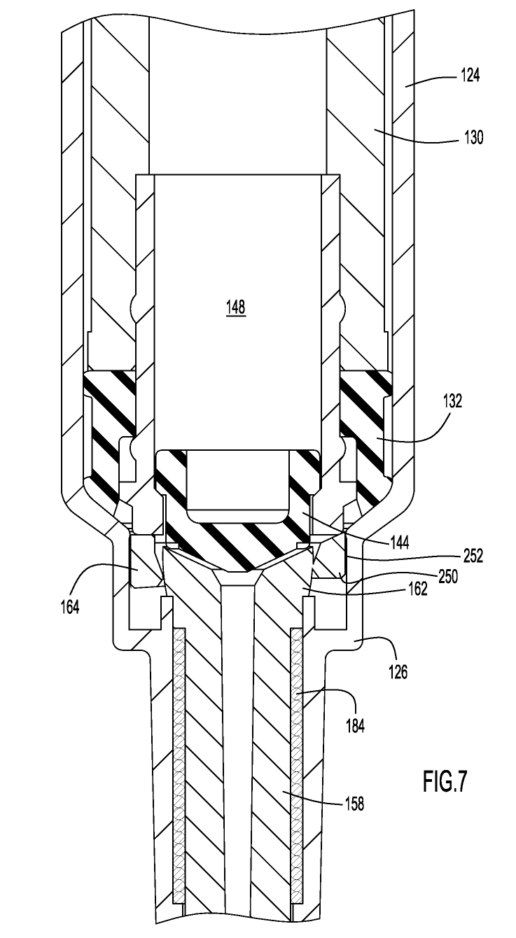

Needle assembly 156 includes a needle stem 158 that connects with a syringe needle 160 and is affixed within the distal end extension 126 of the barrel such that the needle 160 extends from the distal end of the barrel prior to and during use. The needle stem 158 and needle 160 preferably releasably engage with each other via any suitable fluid tight engagement. In an example embodiment, the releasable engagement between the needle stem and the needle is a threaded engagement, where the needle stem includes a male threaded configuration and the needle includes a female threaded connector or needle hub to releasably connect with the needle stem. This connection, which is depicted in FIGS. 1 and 4-7, differs from conventional syringe needle connections, such as Luer Lock configurations. In addition, this threaded configuration provides an easy, universal connection with needles of various gauges and types. However, it is noted that the needle can also be attached to the needle stem in any other suitable releasable or non-releasable manner. Additional examples for releasably engaging a needle including a needle holder or needle hub with the needle stem are described below.

A cavity 157 extends axially from a proximal end of the needle stem 158 to the connection point with the needle 160 in order to facilitate fluid communication between the needle and fluid cavity 150 within the barrel. In addition, cavity 157 includes a widened portion 159 at the proximal end of needle stem 158 that is frusto-conical in configuration and widens toward the proximal end of the barrel so as to be generally aligned and complimentary with the frusto-conical surface 145 of the plunger 130. As noted above, when the plunger is depressed toward the needle assembly, the frusto-conical surface 145 of plug 144 generally aligns and engages with widened portion 159. When the plunger plug is brought toward and fully engages widened portion 159 of the needle assembly, notch 146 defined on plug 144 ensures a fluid flow path exists between fluid cavity 150 and needle assembly cavity 157 and reduces or eliminates any build-up of hydraulic pressure within the fluid cavity.

The needle stem 158 further includes a radially extending flange 162 at its proximal end that is suitably dimensioned to engage with a block or ledge 121 disposed along an interior surface of the distal end extension 126 in order to limit movement of the needle assembly distally beyond ledge 121 during depression of the plunger toward the distal end of the barrel.

A stem ring 164 is secured to and extends radially from the flange 162 of needle stem 158 to engage with the interior wall surface of distal end extension 126 of the barrel. A portion of stem ring 164 is preferably formed or molded as a part of flange 162 and needle stem 158 and includes notched, webbed or scored sections 165 at the connection point of stem ring 164 and needle stem 158, where the scored sections are formed on opposing surfaces of both the needle stem flange and the stem ring. The scored sections 165 define a thin membrane or reduced material section that is torn or broken during operation of the syringe to facilitate retraction of needle assembly 156 in the manner described below. The stem ring 164 is preferably dimensioned to facilitate a partial sliding of a broken portion of the stem ring along the interior wall surface of the barrel when the plunger is depressed to engage with needle stem 158 as described below.

The stem ring 164 includes an inner first ring portion 250 that is connected with flange 162 and an outer second ring portion 252 that connects with and extends radially outward from the first ring portion 250. The first ring portion 250 is preferably molded with so as to be integral or a single component with needle stem 158 and flange 162.

The inner first ring portion 250 of stem ring 164, needle stem 158 and flange 162 are preferably constructed of a suitable medical grade polymer material that has sufficient hardness, strength and durability to connect with a needle and retract with the needle into the plunger cavity in the manner described below during operation of the syringe. Examples of polymer materials suitable for forming the stem ring, needle stem and flange are polystyrene, polycarbonate and acrylonitrile butadiene styrene (ABS).

The second ring portion 252 is constructed of a different polymer material in relation to the first ring portion 250. In particular, the second ring portion is preferably constructed of a suitable polymer material having a lower hardness (e.g., measured as a Shore A or Shore D durometer hardness value) and greater flexibility in comparison to the first ring portion so as to facilitate compression and/or flexing of the second ring portion during movement of the stem ring within the barrel as described below. Example polymer materials that can be used to form the second ring portion include medical grade thermoplastic elastomers, preferably latex-free thermoplastic elastomers (e.g., polyurethane elastomers). The second ring portion is secured to the first ring portion in any suitable manner that prevents separation of the first and second ring portions from each other during use of the syringe. In an example embodiment, the second ring portion is formed with the first ring portion in a co-molding process that secures the two different material portions together.

The size of the first and second ring portions can be selected based upon a selected syringe embodiment. However, it is noted that the radial dimension of material forming each of the first and second ring portions (i.e., the difference between the outer radius and inner radius of each ring portion) is selected such that the radial dimension of material forming the second ring portion is no greater than about 50% of the radial dimension of materials forming the entire stem ring (i.e., the difference between the outer radius of the second ring portion and the inner radius of the second ring portion). In particular, the radial dimension of material forming the second ring portion is preferably from about 1% to about 50%, and more preferably from about 5% to about 10%, of the radial dimension of the entire stem ring.

The diameter of the entire stem ring is slightly larger than the diameter of the interior wall surface of distal end extension 126 at the location where the stem ring engages the barrel such that the second ring portion 252 of stem ring 164 is slightly compressed or is flexed slightly toward or away from the plunger proximal end while maintaining sliding contact or fluid-tight engagement with the barrel so as to ensure an effective fluid tight seal. The dimensions of the stem ring are further selected to provide a compression fit/fluid tight seal at the stem ring/barrel interior wall interface while facilitating a sliding of the stem ring with the barrel interior wall surface when the plunger is completely depressed within the barrel.

A radial protrusion or annular shoulder 166 is disposed along the interior surface and near the proximal end of the distal end extension 126 of the barrel. The shoulder 166 forms a retaining ledge that engages with one or both of the first ring portion 250 and the second ring portion 252 of stem ring 164 to prevent movement of the needle assembly toward the proximal end of the barrel while the stem ring remains attached with needle stem 158. A resilient member 184 (e.g., a coil spring) is disposed between the flange 162 of needle stem 158 and an interior ledge 186 disposed on the interior surface of the distal end extension 126 at a location between ledge 121 and the distal end of the barrel. When the needle assembly 156 is press fit into the distal end extension 126 of the barrel (as described below) such that stem ring 164 is extended distally beyond the radial protrusion 166 (retaining ledge) of the barrel, resilient member 184 is compressed to bias the needle assembly toward the proximal end of the barrel.

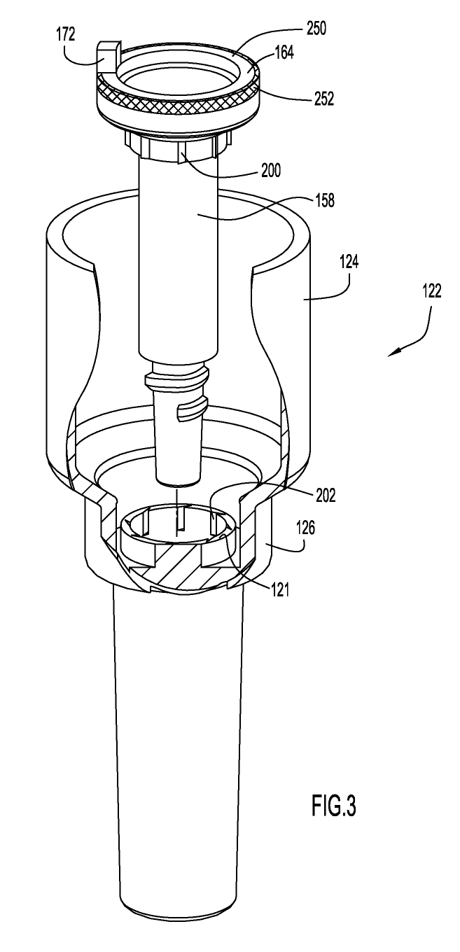

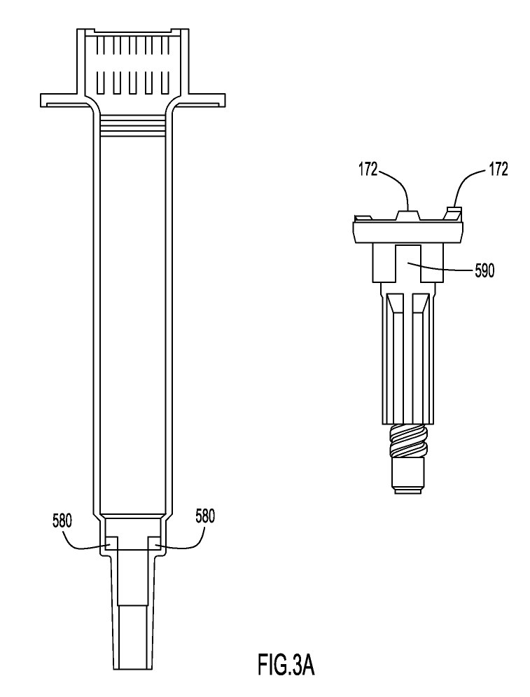

Referring to FIG. 3, the needle stem and barrel include complimentary protrusions and grooves that facilitate easy assembly of the needle stem within the barrel and further limit or prevent any rotation of the needle stem with respect to the barrel after assembly and during operation of the syringe. In particular, needle stem 158 includes a set of protrusions or teeth 200 extending transversely from the outer surface of the needle stem at a location proximate the stem ring 164. The teeth are spaced around the periphery of the needle stem and are aligned to correspond with complimentary grooves 202 disposed along the interior barrel wall surface at the distal end extension 126 and at a location generally corresponding with the barrel ledge 121. As can be seen from FIG. 3, the teeth 200 and complimentary grooves 202 are configured to facilitate easy assembly of needle stem 158 within barrel 122, where the needle stem rotates as necessary upon insertion within the barrel until the teeth are appropriately aligned with the grooves and the needle stem is forced in place within the barrel distal end section. Once needle stem 158 is assembled within barrel 122 and the teeth 200 engage with the grooves 202, the needle stem is prevented from rotating with respect to the barrel during operation of the syringe (e.g., when attaching or removing a needle from the needle stem, and during retraction of the needle assembly within the plunger).

Alternatively, the syringe can be provided with other needle stem and barrel configurations that provide a locking engagement between the needle stem and the barrel. For example, the needle stem and barrel can include suitable complimentary protrusions and/or grooves to achieve a configuration that prevents any rotational movement of the needle stem within the barrel when the needle stem is suitably mounted within the barrel.

In one example configuration shown in FIG. 3A, the barrel includes at least one protrusion at a similar location as complimentary grooves 202 shown in FIG. 3. In particular, the barrel includes two inwardly extending members or blocks 580 spaced apart from each other (e.g., at diametrically opposed locations) along the internal barrel wall periphery. The needle stem includes cut-out sections 590 along its periphery at a location below the stem ring and which correspond with the blocks 580 provided within the barrel. Upon assembling the needle stem within the barrel, the needle stem is aligned such that the cut-out sections 590 of the needle stem align with and receive the blocks 580 of the barrel, thus preventing rotational movement of the needle stem with respect to the barrel.

The syringe 120 is designed so that complete depression of plunger 130 within barrel 122 facilitates a displacement of plunger plug 144 from the plunger and also a tearing or breaking away of the stem ring 164 from flange 162 of needle stem 158 to facilitate retraction of the needle stem and the needle 160 into the retraction cavity 148. In this embodiment, stem ring 164 of needle stem 158 includes at least one raised ridge 172 that extends from a proximal end of the needle stem and is configured to make initial contact with annular distal edge 178 of the plunger when the plunger is displaced within the barrel. While only one ridge 172 is depicted in FIGS. 4-7, it is noted that any selected number of ridges can be provided at any suitable locations and suitably spaced from each other along the proximal end of the needle stem that faces the plunger (see, e.g., the plurality of raised ridges 172 shown in the embodiment of the needle stem of FIG. 3A). The annular distal edge 178 of the plunger is generally planar in configuration. However, it is noted that the plunger distal end can include any one or more protrusions like the needle stem or, alternatively, have an angled contour to form an apex.

Plunger resilient seal 132 is further designed and suitably dimensioned and positioned around the plunger proximate the plunger distal end such that, upon complete depression of the plunger within the barrel, a distal end of the resilient seal engages and compresses slightly against a narrowing portion 190 of the interior barrel wall that defines a transition between main body portion 124 and distal end extension 126. The resilient seal does not slide with respect to the plunger, but rather compresses slightly against the barrel wall narrowing portion at the end of the plunger stroke that initiates needle retraction, and this serves to further minimize or eliminate “dead” space within the fluid cavity 150 as well as to displace any residual fluid from the fluid cavity into needle stem cavity 157.

The needle stem can be designed such that the ridge (or ridges) is aligned in any selected orientation with respect to the plunger distal end. The stem ring ridge(s), plunger and annular distal end of the plunger and needle stem are suitably dimensioned in the longitudinal direction of the syringe and further suitably aligned with each other to facilitate engagement of stem ring ridge(s) 172 and/or other proximal end surface portions of stem ring 164 with annular distal edge 178 of the plunger and plunger plug 144 with needle stem 158 upon complete depression of the plunger, which in turn facilitates a tearing or breakage of stem ring 164 from needle stem flange 158 at the scored sections 165 and a forcing of plunger plug 144 from its frictional engagement with plunger ledge 141 to initiate retraction of needle assembly 56 along with the plunger plug into retraction cavity 48 of the plunger.

Assembly of the syringe is achieved in a similar manner as the other syringe embodiments described above. Resilient member 184 and then needle assembly 156 (with or without the needle 160) are inserted into opening 123 at the proximal end of barrel 122, through main body portion 124 and into the distal end extension 126. As the stem ring 164 of needle stem 158 encounters the retaining ledge or annular shoulder 166 of the barrel, the first ring portion 250 and/or the second ring portion 252 of stem ring 164 is compressed and/or flexed slightly and forced distally beyond the shoulder 166 in a snap-fit engagement. Once stem ring 164 is forced distally beyond shoulder 166, the needle assembly 156 is locked in place within the distal end extension 126, and resilient member 184 is compressed to bias the needle assembly proximally within the syringe. The second ring portion 252 of stem ring 164 remains compressed or flexed to a selected degree between flange 162 and the barrel interior wall surface in this locked configuration and provides an effective fluid tight seal at its compressed fit contact point with the barrel.

Plunger 130 is assembled by inserting plunger plug 144 into the plunger such that the extending portion 147 of the plug frictionally engages plunger ledge 141 located at the open distal end of the plunger and a distal portion of the plunger plug (including plug surface 145 and notch 146) extends from the plunger. The plunger plug can be inserted directly into the open distal end of the plunger to its frictional fitting position. In the embodiment depicted in FIGS. 1-8, the proximal end of the plunger, including thumb pad 138 and flange 139, is a single molded or formed part. Alternatively, the plunger plug can first be inserted through an open proximal end of the plunger (not shown in figures) and pushed into its frictional fitting position prior to sealing this open end with the flange and thumb pad.

After assembly of the plunger, the plunger is inserted into opening 123 of barrel 122 and is axially displaced a suitable distance to facilitate use of the syringe. The needle 160 may be connected with needle stem 158 prior to insertion of the needle assembly into barrel 122 (e.g., via the threaded engagement as depicted in FIG. 3 or in any of the releasable needle engagements described in further detail below). Alternatively, needle 160 may be connected with the connecting portion of needle stem 158 after securing the needle stem within the barrel. Once secured, needle 160 protrudes from the opening at the distal end of the barrel after assembly to facilitate use of the syringe.

In operation, the distal end of the plunger is displaced a suitable distance toward the proximal end of the barrel to draw fluid from needle 160 into fluid cavity 150. Upon injection of the needle into an injection site, the plunger is then depressed toward the distal end of the barrel to force fluid from cavity 150 and through needle 160. Referring to FIGS. 4-7, as the plunger is further depressed within the barrel, the frusto-conical surface 145 of plunger plug 144 moves into the widened portion 159 of central cavity 157 of needle stem 158 to force any remaining fluid through the needle prior to retraction (thus reducing “dead” space between the engaging portions of the plunger and needle assembly). The notch 146 on the plunger plug provides a fluid channel for fluid to continue to flow into central cavity 157 even when plunger surface 145 is substantially in contact with the widened cavity portion 159. In addition, ridge(s) 172 of stem ring 164 initially engages a portion of annular distal edge 178 of the plunger to initiate a tearing or breaking away of stem ring 164 from needle stem 158 along the scored sections 165. Approximately contemporaneously, surface 145 of plunger plug 144 engages needle stem 158 within the widened cavity portion 159, and such continued depression of the plunger toward the needle assembly overcomes the frictional force holding the plunger plug within the plunger, forcing the plunger plug toward the proximal end of the plunger and into retraction cavity 148.

Complete depression of the plunger within the barrel further forces plunger annular distal edge 178 against surface portions of first ring portion 250 and/or second ring portion 252 of stem ring 164, causing a section of the first ring portion 252 of the stem ring that has already broken away from flange 62 to slide distally a short distance along the interior wall of the barrel so as to become oriented at a slight angle with respect to the central axis of needle stem 158. In addition, the portion of the stem ring that has not broken away is prevented from moving distally until it has become broken away from the needle stem. This sliding of the broken portion of the stem ring 164 along the interior wall of the barrel, in combination with the continued pressure applied by the fully depressed plunger to the stem ring, results in a progressive tearing or breakage of the stem ring in both directions along scored sections 165 until the stem ring is fully separated from tab 162. In addition, the forced engagement of plunger plug 144 with needle stem 158 results in further movement of the plunger plug from its frictional engagement with the distal end ledge 141 of the plunger, resulting in dislodging of the plunger plug from the plunger. Plunger resilient seal 132 compresses slightly against the narrowed portion 190 of the barrel interior wall to force residual fluid into the needle assembly, while plunger plug notch 146 provides a fluid flow path for such fluid when plunger surface 145 engages with needle stem 158 within widened cavity portion 159.

The use of a polymer material having a lower durometer and greater flexibility for the second ring portion 252 in relation to the polymer material used to form the first ring portion 250 of the stem ring 164 results in an enhanced fluid seal being achieved at the stem ring/interior barrel wall surface interface in comparison to using a single polymer material for the entire stem ring. In addition, the greater flexibility provided to the stem ring at the interface with the barrel wall surface reduces the contact pressure between stem ring and interior barrel wall surface and facilitates distal movement of a portion of the stem ring and progressive tearing or breakage of the stem ring from the needle stem with a reduction in requisite force to be applied to the plunger in order to achieve retraction of the needle into the syringe.

The design of the syringe can be configured such that the stem ring 164 is completely separated from needle stem 158 immediately prior to, substantially simultaneously with, or immediately after the complete dislodging of plunger plug 144 from the plunger. As shown in the series of FIGS. 4-7, the initial dislodging and axial displacement of plunger plug 144 begins just prior to initial contact between stem ring ridge 172 and plunger distal edge 178 and partial breakage of stem ring 164 from needle stem tab 162. However, the syringe can also be designed such that initial and partial breakage of the stem ring occurs prior to any initial dislodging and axial displacement of the plunger plug. Once complete separation of the stem ring from the tab of the needle stem and at least a partial dislodging of the frictional engagement between the plunger plug and the plunger is achieved, resilient member 184 forces needle stem 158 and needle 160, along with plunger plug 144, proximally into the retraction cavity 148 within the plunger.

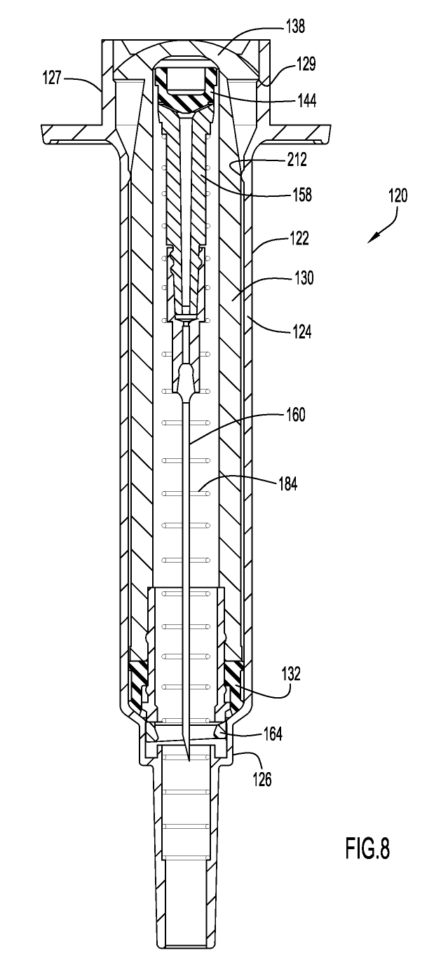

As can be seen from FIG. 8, when plunger 130 has been fully depressed within barrel 122 and retraction of needle assembly 156 has occurred, flange 139 of the plunger extends slightly into the extended barrel portion 127 and is locked within annular groove 129. In this locked position, removal of the plunger from the barrel is prevented.

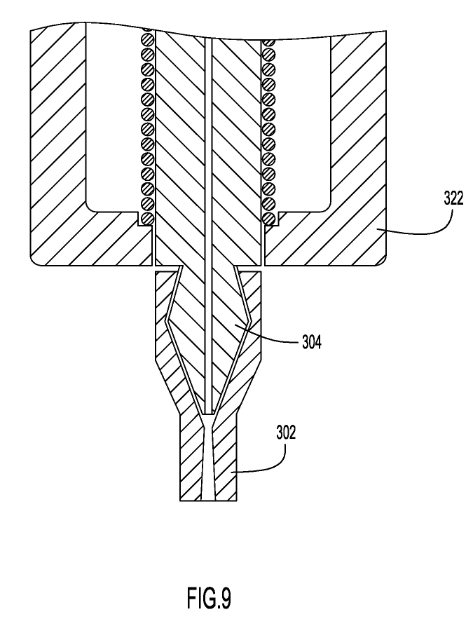

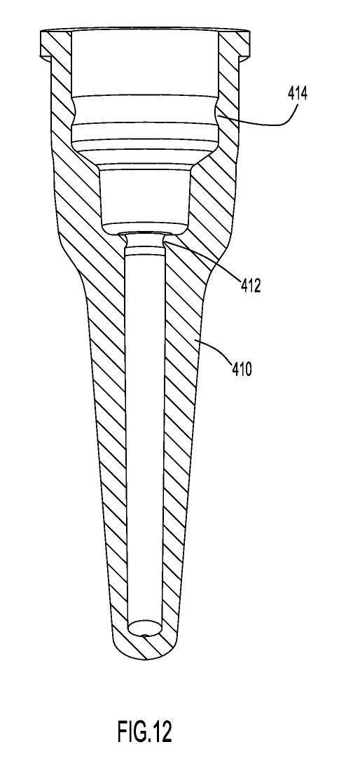

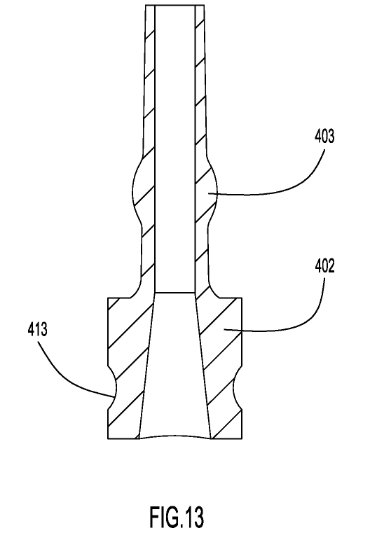

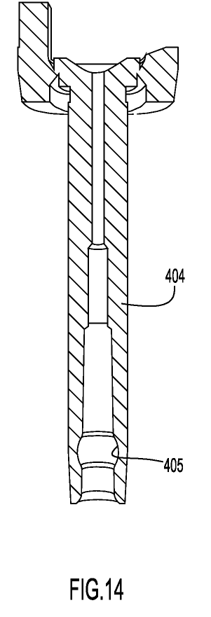

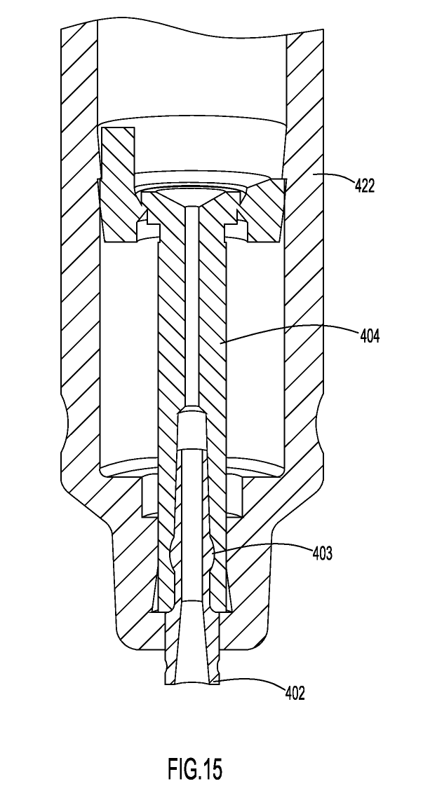

As noted above, the needle can be releasably attached or affixed to the needle stem via a threaded engagement as shown in FIG. 3. In other example embodiments, the needle can be attached to the needle stem in any other suitable manner, for example using snap-tight locking arrangements such as are shown in FIGS. 9-20. It is noted that, in each of these embodiments, the needle retraction features in relation to the needle stem, stem ring, etc. are operable in the same or similar manner as that described above for the previous embodiment.

In FIG. 9, a snap-tight needle connection is shown in which a needle holder or needle hub 302, which attaches directly to a needle (e.g., via adhesive bonding), is configured to be fit over a distal end portion of a needle stem 304 to facilitate a snap-tight locking engagement between needle hub 302 and needle stem 304. The needle stem 304 extends from barrel 322 and includes bumps or protrusions along its periphery which correspond with grooves in the needle hub 302, and the needle hub 302 is formed of a suitably resilient material with sufficient flexibility to facilitate slight expansion of the needle hub as it rides over the protrusions of the needle stem, where the needle hub snaps back to its original position after riding over the needle stem protrusions to achieve a snap-tight lock between the needle stem and needle hub. As can be seen in FIG. 9, the bumps of the stem 304 form a peak or apex on either side of the stem forming a convex “V” shape, and the grooves of the hub 302 have corresponding concave “V” shapes.

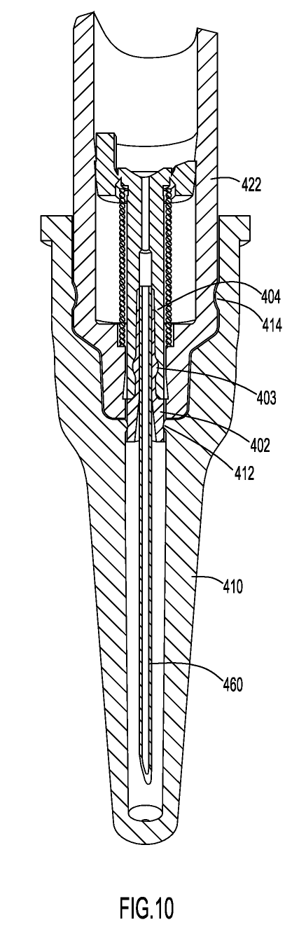

In FIG. 10, a needle holder or needle hub 402 connects with needle stem 404 by insertion of a proximal end of the needle hub into an opening at a distal end of the needle stem, where a snap-tight engagement is achieved when a bump or protrusion 403 along needle hub 402 engages with a corresponding groove 405 within the needle stem 404. The needle stem 404 flexes slightly during insertion of the needle hub 402 into the needle stem to facilitate a connection. In addition, a needle cap 410 is secured around needle 460 and needle hub 402. The needle cap 410 is releasably secured to needle hub 402 (via a bump 412 provided within the needle cap which engages in a snap-tight fit to needle hub 402 at a corresponding groove 413 extending around the needle hub as shown in FIGS. 10, 11 and 13) and also releasably secures to the barrel 422 (at a snap-tight engagement between a bump 414 provided within cap 410 and a corresponding groove along the exterior barrel wall as shown in FIG. 10).

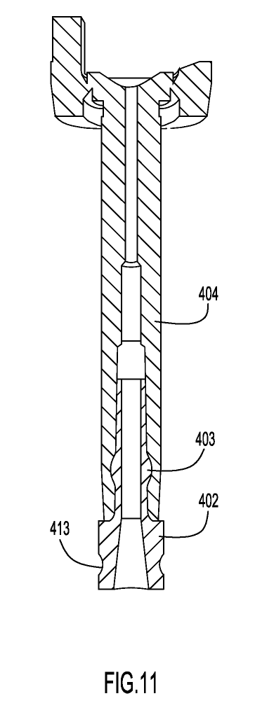

In the embodiment of FIG. 10, the needle hub 402 can first be secured to cap 410 so as to shield the needle 460. The needle hub/cap combination can then be secured to the syringe at the needle stem 404. The distal end of the needle stem 404 is recessed within the barrel, and an elongated section (including bump 403) forming the proximal end of needle hub 402 is inserted within the opening at the distal end of the barrel 422 so as to connect with needle stem 404 (with bump 403 engaging groove 405). When this connection is achieved, the needle cap 410 is also secured around barrel 422 (where bump 414 engages the barrel groove). The syringe of FIG. 10 is further suitably designed to facilitate removal of cap 410 without removal of needle hub 402 from needle stem 404. In particular, once the needle hub 402 is connected with the needle stem 404, the snap-tight holding force (at bump 403 and corresponding needle stem groove 405) between the needle hub and needle stem is greater than the force required to separate the needle cap 410 from both the barrel 422 (at bump location 414) and the needle hub 402 (at bump location 412). FIGS. 11-15 provide additional views (in isolation and in combination with each other) of the needle stem, needle hub and needle cap.

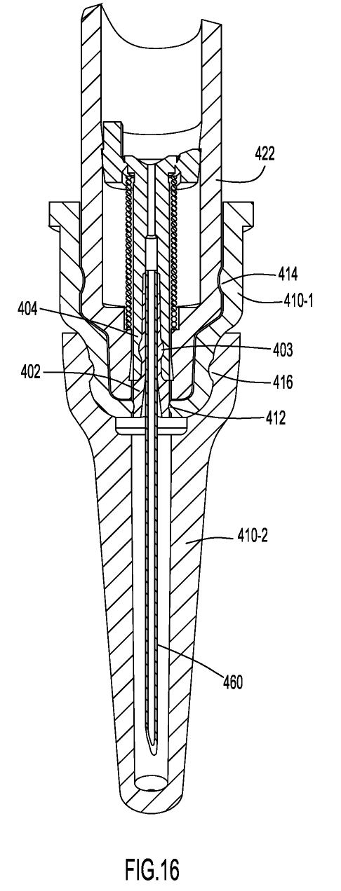

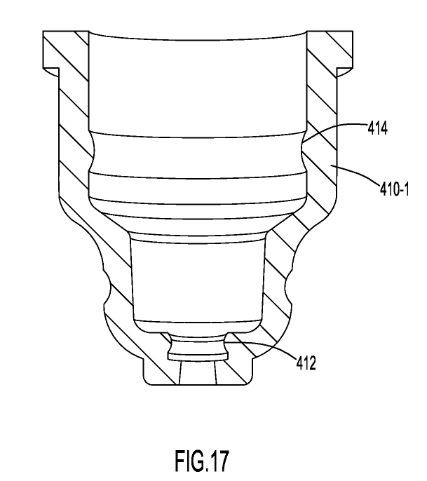

The embodiment of FIGS. 16 and 17 is similar to that shown in FIGS. 10-15, with the exception that the needle cap has a two-piece construction. In particular, the needle cap includes a first part or base 410-1 that secures to needle hub 402 (via bump 412) and barrel 422 (via bump 414). However, the base 410-1 does not cover or shield the needle 460. The needle cap further includes a second part or shield 410-2 that surrounds the needle 460 and connects with base 410-1 via a bump 416 disposed within shield 410-2 that corresponds with a groove disposed along base 410-1.

The two piece needle cap construction shown in FIGS. 16 and 17 facilitates easy assembly of the needle to the needle hub during syringe manufacture. For example, the needle hub 402 can be connected with base 410-1, the needle 460 can then be secured to the needle hub (e.g., using a suitable adhesive), followed by securing shield 410-2 to base 410-1 so as to cover the needle. The two piece needle cap with needle hub can then be attached to the barrel and needle stem 404 (as shown in FIG. 16). When the needle is ready for use, the two piece needle cap can be removed as a single part from barrel 422 by disengaging the engaging bump connections 412, 414 while needle hub 402 remains secured to needle stem 404 at the engaging bump connection 403. Alternatively, the shield 410-2 can be removed while leaving the base 410-1 connected with the barrel and the needle hub 402. In this case, the base 410-1 can be designed such that, during retraction of the needle 460 within the barrel 422 (in the manner described above for the embodiment of FIGS. 1-8), the retraction force applied by the spring to the needle stem 404 overcomes the locking engagement at bump connection 412 such that the hub 402 disengages from the base 410-1 during retraction.

The needle attachment embodiments described above facilitate interchanging of one needle for another during use of a syringe. The snap-tight fit of the bump/groove configurations provides a fluid tight seal between needle hub and needle stem. In addition, these embodiments facilitate using a blunt or fill spike to fill the syringe with fluid, and then replacing the fill spike with a finer gauge needle for use in needle injections. In particular, a blunt or fill spike refers to a hypodermic (i.e., hollow) needle having a larger outer diameter (and a lower hypodermic needle gauge number) in relation to a hypodermic injection needle used for intravenous injection of fluids into a patient. The blunt or fill spike facilitates quick and easy filling of the syringe with fluid, and the syringes of the invention are designed to facilitate easy replacement of the fill spike with an injection needle so as to facilitate use of the syringe for injection of fluids from the syringe into a patient's body.

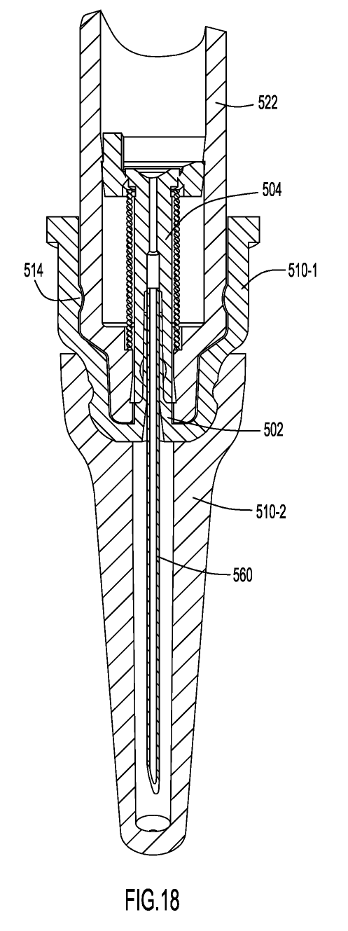

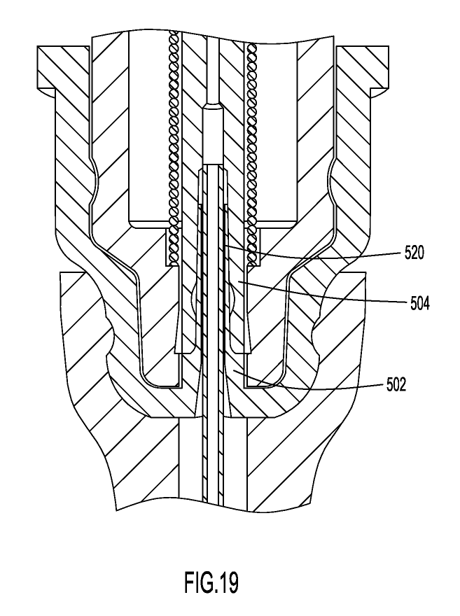

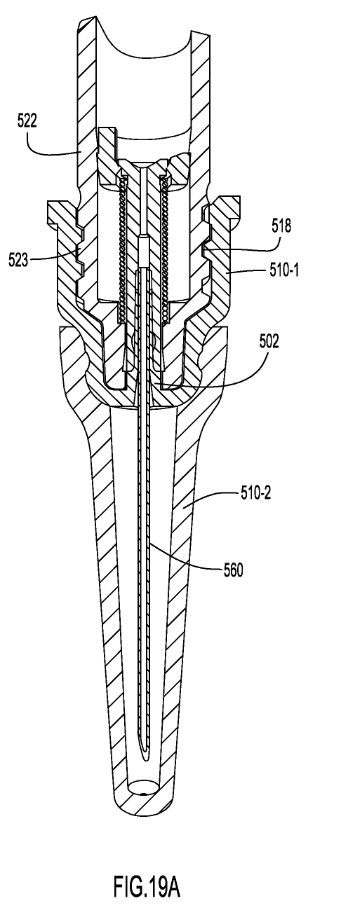

Referring to the embodiment of FIGS. 18 and 19, a blunt or fill spike 560 is connected with a spike hub or holder 502 which is inserted within needle stem 504. A two piece needle cap construction similar to that described above and depicted in FIG. 16 is connected with spike holder 502.

The needle stem 504 in the embodiment of FIG. 18 is similar to the previously described embodiments of FIGS. 10-17, including a two piece needle cap construction. However, the spike holder 502 differs from the previous needle holder or needle hub embodiments in that it does not include any protrusion or bump that provides a snap-tight locking engagement between the spike holder and needle stem. Instead, the elongated proximal end 520 of spike holder 502 extends within needle stem 504 and provides a slight friction and fluid-tight engagement between exterior wall surface portions of the spike holder and interior wall surface portions of the needle stem (see FIG. 19).

In addition, the base 510-1 and spike holder 502 are connected to form a single, integral unit. The base 510-1 connects with the barrel 522 in a similar manner as the previous embodiment of FIG. 16 (with bumps 514 on base 510-1 connecting with complementary grooves on the barrel 522). In addition, the shield 510-2 connects with the base 510-1 in a similar manner as the previous embodiment (with bumps 516 of shield 510-2 connecting with complementary grooves on the base 510-1). The spike is exposed for use with the syringe by removing the shield 510-2 from the base 510-1. After use, the shield 510-2 can be re-connected with the base 510-1 and the entire two piece needle cap assembly with spike and spike holder can be removed from the syringe. This facilitates filling of the syringe quickly using the spike, followed by easy separation of spike holder 502 from needle stem 504 and removal of the needle cap/spike holder from the syringe so as to be replaced by a needle (e.g., using a two-piece needle cap design similar to the embodiment of FIG. 16).

In the embodiment of FIG. 19A, a two-piece cap arrangement with fill spike is shown that is similar to that shown in FIGS. 18 and 19, with the exception that a threaded attachment is provided between base 510-1 and barrel 522 (with male threads 518 provided on base 510-1 and corresponding female threads 523 provided on barrel 522) to facilitate attachment of the needle cap to the base during filling of the syringe with fluid via spike 560. The spike holder 502, which is integral with base 510-1, is free to rotate with respect to the needle stem 504 during connection of the base 510-1 with barrel 522, while frictionally engaging with the needle stem so as to maintain a fluid-tight seal between the spike holder 502 and needle stem 504 after the base 510-1 has been secured to the barrel 522.

The blunt or fill spike can be formed of any suitable medical grade materials, such as medical grade polycarbonate, polystyrene or other suitable polymer materials.

The blunt or fill spike configuration can also be modified to include filter media disposed between portions of the fill spike fluid path so as to facilitate filtering of fluid passing through the fill spike into the syringe. The filter media can be formed of any suitable material that is capable of filtering particulate material or other matter of selected dimensions from the fluid prior to entering the syringe and prior to injection in a subject.

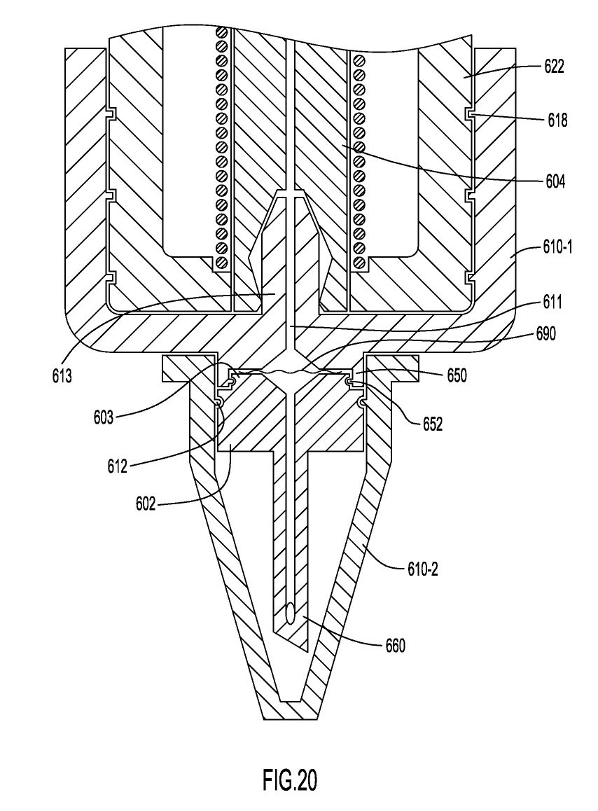

An example design of a blunt fill needle connection to a retractable syringe is depicted in FIG. 20. In particular, a two-piece cap design is provided that compresses or sandwiches a filter media at a connection point between each cap piece, where a base cap piece 610-1 connects to the barrel 622 (e.g., via a threaded connection including male threads 618 on the base 610-1 and corresponding female threads on the barrel 622) and includes a portion 613 that extends within the needle stem 604. The portion of base 610-1 extending within the needle stem 604 has a fluid pathway 611 that communicates with the fluid pathway of the needle stem 604. A shield portion 610-2 connects with a spike holder 602 including a fill spike 660 via bumps 612 disposed on internal surfaces of the shield portion 610-2 and corresponding grooves of the spike holder 602. The spike holder 602 and fill spike 660 can be integrally formed as a single unit (e.g., as shown in FIG. 20) or, alternatively, as separate members secured together in a fluid tight engagement.

The base 610-1 includes a seat section 650 protruding from the distal end of the base, where the seat section 650 includes fluid pathway 611 and is designed to receive a proximal end of the spike holder 602 such that the fluid pathway 611 communicates with the fluid pathway of spike 660. The fluid pathway 611 further extends through portion 613 so as to provide a fluid pathway between the spike 660 and the needle stem 604. The seat section 650 includes a recessed portion and a rim surrounding the recessed portion, and the spike holder 602 includes a stepped portion 603 at its proximal end that has a reduced cross-sectional dimension in relation to the portion of the spike holder that connects with the shield portion 610-2 (via the bumps 612 and corresponding grooves), where the stepped portion 603 is configured to fit within the recessed portion of the seat section 650 when the spike holder 602 and shield portion 610-2 engage with the seat section 650 so as to connect the spike to the syringe.

In addition, the rim of the seat section 650 includes bumps 652 that are configured to engage with corresponding grooves on the stepped portion 603 of the spike holder 602 when the spike holder engages the distal end of the base 610-1. A suitable filter media material 690 is provided at a location between the engaging surfaces of the seat section 650 and stepped portion 603 of the spike holder 602. For example, the filter media can be provided over the stepped portion 603 or, alternatively, over the recessed portion within the seat section 650 such that, upon engagement of the spike holder 602 with the seat section 650 of base 610-1, the filter media 690 is “sandwiched” between the two components to effectively filter materials of selected dimensions from fluids flowing through the spike and into the syringe.

The spike holder 602 initially connects with shield 610-2 via the bump 612 and groove connections as shown in FIG. 20. The base 610-1 is also connected with the barrel 622 via the threaded engagement such that base portion 613 extends within needle stem 604. The shield 610-2 including the spike holder 602 can then be connected with the base 610-1 by inserting the stepped portion 603 of the spike holder 602 into the recessed portion of seat section 650 (with a proximal end portion of shield 610-2 extending around the rim of seat section 650) such that the bump 652 and groove connections of the seat section 650 and spike holder 602 engage. The shield 610-2 can then be removed from the spike holder 602 so as to expose the spike 660 and render the syringe ready for use. After filling of the syringe, the shield 610-2 can be re-connected with the spike holder 602 and the entire assembly (including base 610-1, spike holder 602 and shield 610-2) can be removed from the syringe to facilitate connection of an injection needle with the syringe.

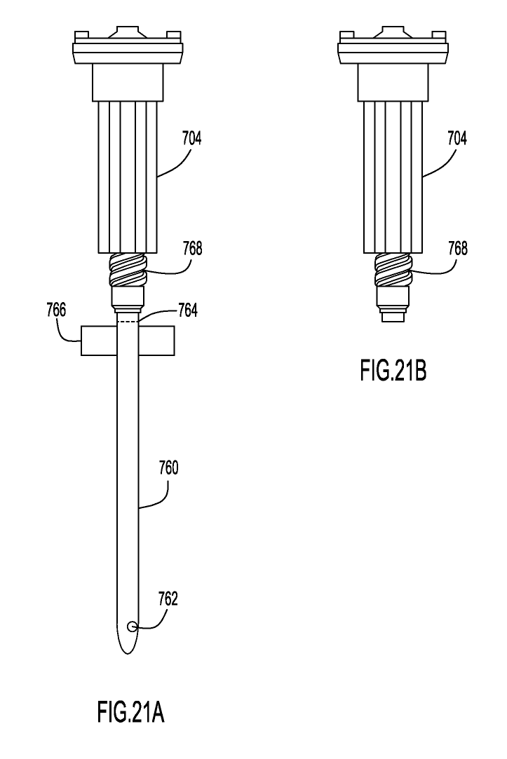

In another embodiment shown in FIG. 21, a needle stem is designed to include a spike that is integral with the needle stem but can be easily removed via a “snap-off” connection. The spike is preferably formed of a suitable plastic material to facilitate easy separation of the spike from the needle stem. The needle stem 704 is similar in design and functionality in relation to the needle stems described for the previous embodiments, with the exception that needle stem 704 includes a spike 760 integrally connected with the needle stem 704. The spike 760 is sealed or enclosed at its distal end but includes a hole 762 along a side near its distal end. The hole 762 prevents coring of a vial into which the spike is inserted and/or prevents material from the vial core from entering the spike and also the syringe during withdrawal of fluids from the vial into the syringe. A scored section 764 is provided on the spike 760 at a location near its proximal end (i.e., at a location at which the spike is integrally formed with the needle stem). This scored section 764 can be a reduced material section that maintains a fluid tight seal at this section but also facilitates easy separation (e.g., by tearing, breaking or snapping removal) of the spike 760 from the needle stem 704 after use of the spike to fill the syringe. Optionally, one or more flanges 766 can also be provided at a location in close proximity with the scored section 764 to facilitate easy breaking or snapping off of the spike 760 from the needle stem 704.

After removal of the spike 760 from the needle stem 704, any suitable needle engaging structure (such as the types described above) can then be utilized with the needle stem. In the embodiment of FIG. 21, the needle stem 704 includes a threaded section 768 that can facilitate connection with a needle holder (e.g., similar to the needle connection depicted in the embodiment of FIGS. 1-8). However, any other suitable needle connection configuration can also be utilized to connect the needle stem with a needle so as to achieve enhanced needle changing and interchangeability for the syringe.

The needle changing/interchangeability features of the invention are not limited to the previous examples. Instead, it is noted that the connections between needle holder and needle stem, and needle holder and needle cap designs can have any suitable designs and configurations that facilitate connection of the needle to the syringe. For example, the bumps and grooves or recesses which achieve the snap-tight and fluid tight engagements can be reversed (e.g., the needle hub or holder can include a groove and needle stem a corresponding bump to facilitate a connection between these two components as shown in FIG. 9). Any one, two or more bumps, protrusions and corresponding grooves and/or ratcheting teeth engagements can be provided to facilitate a snap-tight or other suitable locking connection between needle hub or holder and needle stem. In addition, the snap-tight engagements between needle holder, needle cap portions and barrel can be modified to include threaded engagements between such components (e.g., threaded engagement configurations as described in some of the previous embodiments).

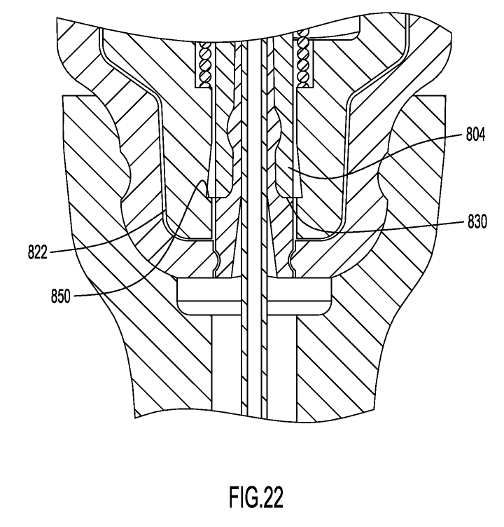

Another important feature in retractable syringes such as the types described above is to prevent the needle stem from moving or “floating” within the barrel during use of the syringe and prior to retraction of the needle within the syringe. As noted above, the barrel of the syringe of the present invention includes a radial protrusion (e.g., retaining ledge or annular shoulder 166 as shown in FIG. 4) disposed along the interior surface and near the proximal end of the distal end extension of the barrel, and this protrusion prevents movement of the stem ring and needle stem toward the proximal end of the barrel prior to tearing away and separation of the stem ring from the needle stem. To prevent the needle stem from moving toward the distal end of the barrel during syringe operation, the needle stem is typically grounded by a ledge disposed within the barrel. For example, FIG. 22 shows that a distal end 830 of needle stem 804 abuts against a ledge 850 formed within barrel 822, such that the barrel ledge 850 effectively grounds the needle stem by preventing movement of the needle stem toward the barrel distal end.

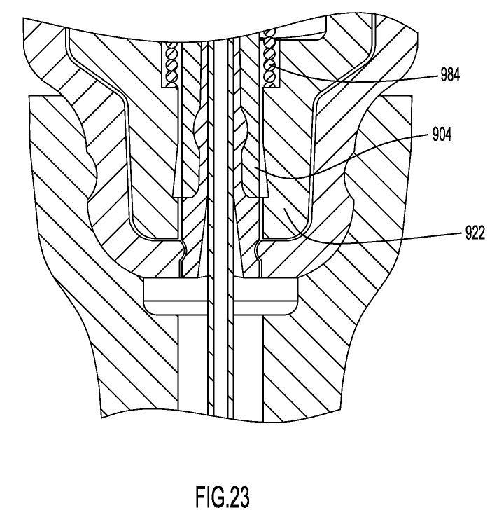

Another mechanism for preventing movement of the needle stem toward the barrel distal end is provided by the present invention. In particular, the spring that biases the needle stem toward the barrel proximal end can be designed and configured with a suitable tension such that the spring itself prevents movement of the needle stem distally (i.e., toward the distal end of the barrel) within the syringe. Such a design facilitates providing a larger diameter or open distal end for the barrel, since no ledge is necessary to ground the needle stem within the barrel. The larger diameter or open distal end for the barrel further facilitates providing a needle holder with larger transverse cross-sectional dimensions, which can facilitate easier design and construction of the needle holder. An example of this embodiment is shown in FIG. 23, where it can be seen that needle stem 904 is capable of protruding through the open distal end of barrel 922. However, spring 984 is designed having suitable tension to prevent distal movement of the needle stem 904 too far toward the barrel distal end so as to prevent “floating” of the needle stem during operation of the syringe. Thus, the tension of the spring acts as a stop or grounding mechanism similar to the ledge 850 of the barrel 822 described in the embodiment of FIG. 22.

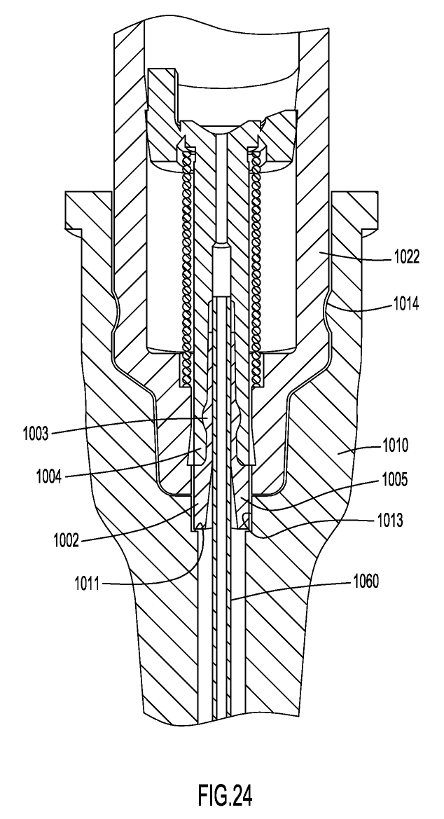

In the embodiment of FIG. 24, a needle cap/needle hub assembly is provided including a single cap 1010 and needle hub 1002 that connects with a needle stem 1004 of a barrel 1022 in a manner similar to the embodiment described above and depicted in FIG. 24, where the cap 1010 includes at least one bump 1014 that engages with a corresponding groove on the barrel 1022 to releasably engage the cap with the barrel and the hub 1002 with needle 1060 includes at least one bump 1003 to engage with a corresponding groove of the needle stem 1004 to engage the needle hub with the needle stem. However, the cap 1010 does not releasably lock with the needle hub 1002. Instead, the needle cap 1010 includes an internal ledge 1011 that is configured to engage with a block section 1005 of the hub 1002 so as to provide a stop to prevent movement of the needle hub away from the needle stem when the needle hub and needle stem are to be engaged with each other. In addition, the needle cap can include one or more splined surfaces at the ledge or at other locations (e.g., splined surface 1013 of cap 1010 as shown in the magnified view of FIG. 24) to provide an enhanced holding force with respect to the needle hub prior to attachment of the needle hub to the needle stem.

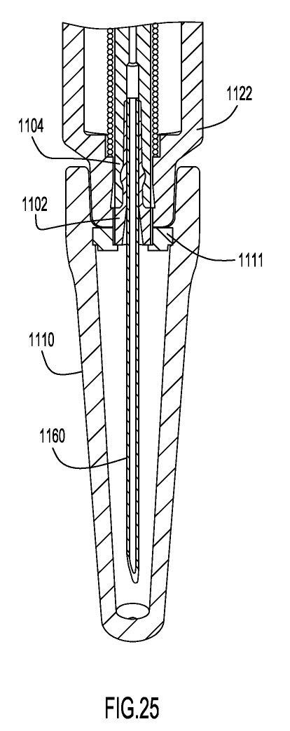

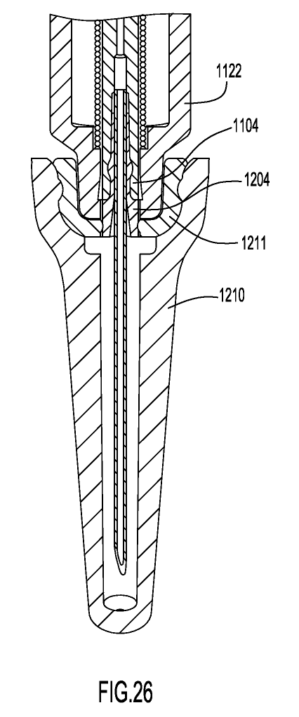

In still another embodiment, shown in FIG. 25, a needle cap 1110 includes a ring 1111 (or other suitable ledge structure) disposed between the cap 1110 and a block section of the needle hub 1102 to facilitate a grounding of the needle hub 1102 within the needle cap 110 and also to stabilize and align the needle 1160 properly for insertion within the needle stem 1104 during assembly of the hub 1102 with stem 1104. The ring 1111 includes a stepped section on its outer periphery that engages with a notched section within the cap 1110 to lock the ring with the cap. In addition the ring 111 includes a distal end section that extends partially over a distal end of the block section of the needle hub 1102. In this embodiment, cap 1110 does not releasably lock with the barrel 1122 but instead frictionally engages with the tapered distal end section of the barrel 1122 during assembly of the needle hub 1102 with the needle stem 1104.

In a modified design to the cap with ring embodiment, which is depicted in FIG. 26, ring 1211 engages with cap 1210 via a bump and groove arrangement disposed between the ring and cap. In addition, the ring 1211 extends around a portion of the tapered distal end section of barrel 1122 so as to be disposed between the cap 1210 and the barrel 1122. Further, the ring 1211 engages with the hub 1102 via a bump and groove arrangement, which is different from the embodiment of FIG. 25 (in which ring 1111 extends partially over the distal end of the block section of hub 1102).

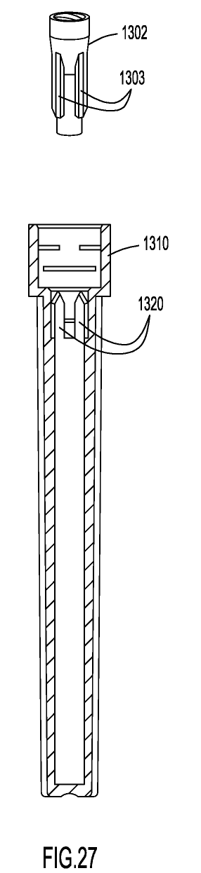

In FIG. 27, another embodiment of a combination needle hub and needle cap arrangement is shown, in which the needle hub 1302 includes female threads to facilitate connection of the needle hub with a needle stem (e.g., a needle stem 158 as shown in the embodiment of FIGS. 1-8. The needle hub 1302 includes ribs 1303 which are aligned in a longitudinal direction of the hub and are spaced from each other along the hub periphery. The needle cap 1310 includes internal grooves 1320 that are aligned to receive the ribs 1303 when the hub is placed within the needle cap. This rib and groove configuration facilitates rotation of the needle hub to threadingly engage with the needle stem by rotation of the needle cap, thus permitting attachment of the needle hub to the needle stem while keeping the needle (which connects with the needle hub via an adhesive or other suitable connection) shielded within the cap. In addition, a portion of the outer peripheral edge portion of the proximal end of the hub can be removed at a selected draft angle to enhance retraction of the needle hub and stem, since this removed portion will prevent the possibility of the spring or resilient member from catching at the hub during retraction.

Thus, the present invention facilitates connection of one or more needles of varying gauges to a retractable syringe with relative ease, in which a cap (or portion of a cap) shields the needle while the needle holder is connected with the needle stem of the syringe. The present invention further facilitates the use of a retractable syringe with a blunt or fill spike, removal of the fill spike and replacement with an injection hypodermic needle (i.e., a needle having a smaller outer diameter than a fill spike that is used for intravenous injections in a patient). The many different embodiments facilitate easy interchangeability of the syringe with different types and sizes of needles while maintaining safe enclosure of the needle during assembly and further while maintaining the retractable features of the syringe.

The present invention further provides an enhanced sealing mechanism for the retractable needle stem ring which engages the internal surface portion of the barrel wall. This two-piece needle stem ring construction renders the assembly of the needle stem within the barrel easier, maintains an effective fluid tight seal and can also possibly reduce the retraction force required to retract the needle stem, needle holder (or needle hub) and needle within the syringe.

While the invention has been described in detail and with reference to a specific embodiment thereof, it will be apparent to one skilled in the art that various changes and modifications can be made therein without departing from the spirit and scope thereof. Thus, it is intended that the present invention covers the modifications and variations of this invention provided they come within the scope of the appended claims and their equivalents.

What is claimed:

1. A syringe comprising:
a hollow barrel including a proximal end with an opening and a distal end configured to receive a syringe needle, wherein a portion between the proximal and distal ends of the barrel defines a fluid cavity to receive and expel fluid from the barrel distal end;
a plunger extending into the proximal end opening of the barrel and axially movable within the barrel toward and away from the barrel distal end, wherein the plunger includes a proximal end and a distal end that extends within the barrel and is movable toward and away from the barrel distal end to facilitate drawing fluid into and forcing fluid from the fluid cavity within the barrel; and
a needle assembly including a section secured within the barrel, the needle assembly including a needle holder, a resilient member biasing the needle holder toward the proximal end opening of the barrel, and a retaining member releasably secured to the needle holder, wherein the retaining member includes a first material that connects the retaining member to the needle holder and a second material that engages an interior wall section of the barrel, the second material having a Shore A or Shore D durometer value that is less than a Shore A or Shore D durometer value of the first material.

2. A syringe comprising:
a hollow barrel including a proximal end with an opening and a distal end configured to receive a syringe needle, wherein a portion between the proximal and distal ends of the barrel defines a fluid cavity to receive and expel fluid from the barrel distal end;
a plunger extending into the proximal end opening of the barrel and axially movable within the barrel toward and away from the barrel distal end, wherein the plunger includes a proximal end and a distal end that extends within the barrel and is movable toward and away from the barrel distal end to facilitate drawing fluid into and forcing fluid from the fluid cavity within the barrel; and
a needle assembly at least partially secured within the barrel, the needle assembly including a needle stem, a resilient member biasing the needle stem toward the proximal end opening of the barrel, a needle holder connectable to the needle stem during assembly of the syringe and that is further securable to a needle, and a cap that is dimensioned to surround the needle secured to the needle holder and a portion of the needle holder during assembly of the needle holder with the needle stem.

3. The syringe of claim 2, wherein the needle holder includes at least one bump section that is aligned to engage with a groove section of the needle stem so as to releasably lock the needle holder with the needle stem.

4. The syringe of claim 2, wherein the cap includes a section that engages with the barrel to releasably lock the cap with the barrel.

5. The syringe of claim 4, wherein the cap section includes at least one bump section that is aligned to engage with a groove section of the barrel.

6. The syringe of claim 4, wherein the cap section releasably engages with the barrel via a threaded connection between the cap section and the barrel.

7. The syringe of claim 4, wherein the cap further includes a bump section that releasably engages with a groove section of the needle holder to releasably engage the cap with the needle holder, and the needle holder includes at least one bump section that is aligned to engage with a groove section of the needle stem so as to releasably lock the needle holder with the needle stem.

8. The syringe of claim 7, wherein the bump and groove sections of the needle holder and needle stem are configured such that the needle holder and needle stem remain engaged when the bump section of the cap is removed from the groove section of the needle holder.

9. The syringe of claim 4, wherein the cap includes a base that releasably locks with the barrel and engages with the needle holder and a shield that covers the needle secured to the needle holder and releasably engages with the base.

10. The syringe of claim 9, wherein the base includes a bump section that releasably engages with a groove section of the needle holder, and the shield includes a bump section that releasably engages with a groove section of the base.

11. The syringe of claim 9, wherein the base and the needle holder are formed as a single, integral unit, and the needle comprises a fill spike having suitable dimensions to facilitate filling of the syringe with fluid prior to an injection needle being connected with the syringe.

12. The syringe of claim 11, wherein the base releasably locks with the barrel via a threaded connection between the base and the barrel.

13. The syringe of claim 9, further comprising a filter media disposed between the base and the needle holder, wherein the base further comprises a section that is in fluid communication with the needle stem to facilitate a flow of fluid filtered by the filter media is through the needle stem and into the syringe.

14. The syringe of claim 1, further comprising a filter media to filter fluids drawn within the syringe.

15. The syringe of claim 1, wherein the needle stem includes a fill spike connected with a distal end of the needle stem to form a single unit, and the fill spike is removable from the needle stem to facilitate connection of the needle stem with the needle holder.

16. The syringe of claim 1, wherein a proximal end of the cap engages with the distal end of the barrel, and the cap includes an internal section that engages with the needle holder to prevent movement of the needle holder toward a distal end of the cap during connection of the needle holder with the needle stem.

17. The syringe of claim 16, wherein the cap includes a ring member at least partially disposed within the cap that surrounds a portion of the needle holder to prevent movement of the needle holder toward a distal end of the cap during connection of the needle holder with the needle stem.

18. The syringe of claim 1, wherein the needle holder connects with the needle stem via a threaded engagement, and the needle holder includes ridges disposed on an external surface of the needle holder and that engage with corresponding internal grooves within the cap such that, after insertion of the needle holder within the cap and placement of the needle holder in contact with the needle stem, rotation of the cap facilitates a threaded connection between the needle holder and the needle stem.

19. A needle assembly for use with a syringe comprising a hollow barrel including a proximal end with an opening and a distal end configured to receive a syringe needle, a portion between the proximal and distal ends of the barrel defining a fluid cavity to receive and expel fluid from the barrel distal end, a plunger extending into the proximal end opening of the barrel and axially movable within the barrel toward and away from the barrel distal end, the plunger including a proximal end and a distal end that extends within the barrel and is movable toward and away from the barrel distal end to facilitate drawing fluid into and forcing fluid from the fluid cavity within the barrel, and a needle stem and a resilient member biasing the needle stem toward the proximal end opening of the barrel, the needle assembly comprising:
a needle holder that is connectable to the needle stem during assembly of the syringe and that is further securable to a needle; and
a cap that is dimensioned to surround the needle secured to the needle holder and a portion of the needle holder during assembly of the needle holder with the needle stem such that the needle is shielded by a portion of the cap when the needle holder with needle is secured to the needle stem of the syringe.

20. A method of securing a needle to a syringe, the syringe comprising a hollow barrel including a proximal end with an opening and a distal end configured to receive a syringe needle, a portion between the proximal and distal ends of the barrel defining a fluid cavity to receive and expel fluid from the barrel distal end, a plunger extending into the proximal end opening of the barrel and axially movable within the barrel toward and away from the barrel distal end, the plunger including a proximal end and a distal end that extends within the barrel and is movable toward and away from the barrel distal end to facilitate drawing fluid into and forcing fluid from the fluid cavity within the barrel, and a needle stem and a resilient member biasing the needle stem toward the proximal end opening of the barrel, wherein the method comprises:
installing a needle holder including a needle secured to the needle holder within a cap such that the cap shields the needle; and
securing the needle holder to the needle stem of the syringe while the cap is shielding the needle.

21. The method of claim 20, wherein the needle holder includes at least one bump section that is aligned to engage with a groove section of the needle stem so as to releasably lock the needle holder with the needle stem.

22. The method of claim 21, further comprising:
releasably securing the cap to the barrel when the needle holder is secured to the needle stem.

23. The method of claim 22, The syringe of claim 4, wherein the cap section releasably engages with the barrel via one of a threaded connection between the cap section and the barrel and a bump section of the cap that is aligned to engage with a groove section of the barrel.

24. The method of claim 20, further comprising:
removing the cap from the barrel while the needle holder and needle stem remain secured to each other.

25. The method of claim 22, wherein the cap includes a base that releasably locks with the barrel and engages with the needle holder and a shield that covers the needle secured to the needle holder and releasably engages with the base.

26. The method of claim 25, wherein the base includes a bump section that releasably engages with a groove section of the needle holder, and the shield includes a bump section that releasably engages with a groove section of the base.

27. The method of claim 25, wherein the base and the needle holder are formed as a single, integral unit, and the needle comprises a fill spike, the method further comprising:
removing the shield from the barrel;
filling the syringe with fluid via the fill spike;
removing the base and needle holder from the syringe; and
installing an injection needle with the needle stem of the syringe.

28. The method of claim 27, wherein a filter media is disposed between the base and the needle holder, and the base further comprises a section that is in fluid communication with the needle stem such that fluid filled within the syringe through the fill spike is filtered by the filter media.

29. The method of claim 20, wherein the needle stem includes a fill spike connected with a distal end of the needle stem to form a single unit, and, prior to securing the needle holder to the needle stem, the method further comprises:
filling the syringe with fluid via the fill spike; and
removing the fill spike from the distal end of the needle stem to facilitate connecting the needle holder to the needle stem.

30. The method of claim 20, wherein the needle holder connects with the needle stem via a threaded engagement, and the needle holder includes ridges disposed on an external surface of the needle holder and that engage with corresponding internal grooves within the cap such that rotation of the cap facilitates the securing of the needle holder to the needle stem via the threaded engagement.
